(19) United States
(12) Patent Application Publication
(10) Pub. No.: US 2005/0220730 A1
(43) Pub. Date: Oct. 6, 2005

(54) Nail polish compositions comprised of nanoscale particles free of reactive groups

(76) Inventors: Alain Malnou, Routot (FR); Martinez Francisco, Chartres (FR)

Correspondence Address: Patrich H. Higgins;Mathews, Collins, Shepherd & McKay, P.A., Suite 306, 100 Thanet Circle, Princeton, NJ, 08540-3674, US

(21) Appl. No.: 10/877,596

(22) Filed: Jun. 25, 2004

(30) Foreign Application Priority Data
Apr. 1, 2004 (FR) 04.03449

(51) Int. Cl. A61K 7/04

(52) U.S. Cl. 424/61

(57) ABSTRACT

Nail polish compositions and method of using a composition for nail polish is provided which composition comprises about 0.01% to 20% by weight of non-aggregated particles which have a mean size between about 5 nm and about 100 nm, a main film forming polymer, a secondary film forming polymer, at least one plasticizer, at least one solvent, wherein the particles do not have reactive groups on a surface, and wherein the particles have a refractive index between about 1.4 and about 1.7.

The Applicants herein claim priority under 35 USC §119 to French National Application No. 04.03449, filed Apr. 1, 2004.

### FIELD OF THE INVENTION

The present invention relates generally to cosmetics and cosmetic formulations for external use on the body; and more particularly to novel long lasting nail polishes. A nail polish composition comprised of nanometric particles is described which exhibits improved properties including better physical resistance, improved gloss, transparency, and viscosity for application.

### BACKGROUND OF THE INVENTION

Cosmetics and their use as decorative applications for the human body have been in existence for centuries and are used extensively throughout the world. As a multibillion-dollar industry, cosmetics have many different applications, including the use of lipstick, eyeliner, facial creams, powders, highlighters, and nail polishes or lacquers. Used primarily by women, the extent of cosmetic use is extraordinary, and has made many of the cosmetics producing and marketing firms very profitable endeavors.

The nail polish sector of the cosmetic industry is extremely competitive. New colors, combinations, including clear formulations and variations thereof are constantly being introduced in an attempt by the formulator to get and hold onto a bigger piece of the market share. The scientific research that is poured into these endeavors is considerable and new products and/or brand names are continually being introduced.

Nail polishes are essentially comprised of pigments and/or dyes that are incorporated and suspended in various solvents and bases. The polishes are also stabilized in the suspension to protect the appearance of the product and to prevent the pigments, during the shelf life of the product, from floating, settling, separating, or striating the polish. Nail polishes are also formulated so that the film formed thereby is tough and durable and adheres to the human nail and will not readily crack, chip, peel, splinter, or become brittle after application and during wear. The toughness should last for an extended period of time, thereby enhancing the durability of the nail polish enamel.

Nail polish compositions generally comprise a main film forming polymer, for example, nitrocellulose or cellulose acetate butyrate, or a combination of one or both of these cellulosic compounds with a polyurethane or other polymeric compound. Secondary polymers such as polyester resins, acrylic resins or condensation resins such as tosylamide, and one or more plasticizers and solvents. Nail polish compositions generally further contain colouring substances, thickeners and additives such as anti-UV, wetting agents, levelling agent(s), hydrating substances, perfumes, and the like. Nail enamels have traditionally included plasticizers, typically a phthalate such as dibutyl phthalate, or camphor, and have also typically included as an adhesion promoter, a polymeric component formed by the condensation polymerization of formaldehyde or other aldehyde, typically an aromatic sulfonamide-aldehyde condensation resin such as o-toluene sulfonamide formaldehyde resin, or a polyester resin such as phthalic anhydride trimellitic anhydride/glycol copolymer.

Nail polishes are generally formulated with an intent to optimise the performance upon application. Desirable properties include a good levelling coat, a short drying time and a very good gloss and with a optimal scratch, wear and chipping resistance as well as a resistance to forming a surface film upon exposure to water and soaps. Nail adhesion is important as well as a proper balance of hardness and flexibility. Extenders improve the balance, but with a loss of gloss and transparency. Nail polish may be formulated to produce a film upon application with a relative low hardness that produces good adhesion without chipping. Wear is further reduced by wax and silicone additives. These type compositions, however, confer significant loss of gloss.

U.S. Pat. No. 5,066,484 to Castrogiovanni et al. discloses a number of formulations including glycerol triesters useful as plasticizers in nail polish compositions. The formulations further comprise a film-forming component, a solvent, pigments and other optional compounds. U.S. Pat. No. 5,145,670 to Castrogiovanni et al. discloses nail compositions comprised of glycerol triacetates and trioctanoates as the plasticizers. U.S. Pat. No. 5,225,185 to Castrogiovanni et al. discloses nail lacquers in which the formulation is free of formaldehyde and phthalate, and the plasticizer is comprised of a copolymer formed by the condensation polymerization of formaldehyde and/or other aldehyde, typically an aromatic sulfonamidealdehyde condensation resin. U.S. Pat. No. 5,227,155 to Castrogiovanni et al. discloses nail lacquers with glycerol triacetylricinoleate and glycerol tribenzoate as the plasticizers.

U.S. Pat. No. 5,133,966 to Khamis discloses nail polish formulations comprising improved pigment suspension systems. More specifically, the suspensions comprise carboxylic acids and their salts with a resin carrier in a buffered coating process that is described as yielding a hydrophobic coating with increased dispersion characteristics. U.S. Pat. No. 5,174,996 to Weber et al. discloses nail enamels with improved flotation, migration, and settlement characteristics, which features are achieved by coating pigments with oxidized polyethylene.

U.S. Pat. No. 5,882,636, describes phthalate-free nail polish enamel that includes a film-forming agent, solvents, one or more colorants, one or more suspending agents, adhesion promoters, and plasticizers. The plasticizers used include adipates, pentaerythrityl tetrabenzoate, 2,2,4-trimethyl-1,3-pentanediol diisobutyrate, pentaerythrityl tetraacetate, and mixtures thereof.

Chemists have previously used colloidal silica in nail polish compositions with or without silane treatment. The nanoscale particles, however, due to the highly active surfaces, have strong interactions between them and as a result, the particles tend to agglomerate during production of the composition. It is very difficult to obtain a homogenous dispersion with nanoscale particles without problems of viscosity, transparency and gloss. In particular, a significant rise of viscosity is often observed that raise problem with application, gloss and strength of the film.

### SUMMARY OF THE INVENTION

The present invention is directed to a nail polish composition comprising about 0.01% to about 20% by weight of non-aggregated particles which have a mean size between about 5nm and about 100 nm, wherein the particles do not have reactive groups on a surface.

In addition, the current invention is directed to a method of using a composition for nail polish which composition, for example, comprises about 0.01% to about 20% by weight of non-aggregated particles which have a mean size between about 5 nm and about 100 nm, a main film forming polymer, a secondary film forming polymer, at least one plasticizer, at least one solvent, wherein the particles do not have reactive groups on a surface, and wherein the particles have a refractive index between about 1.4 and about 1.7.

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference.

Herein disclosed is the unexpected discovery that the use of certain nanoscale particles, particularly oxides with no active/reactive surfaces detectable by infrared measurements, even at low concentrations confer valuable properties to the nail polish compositions without otherwise introducing viscosity problems. The term active/reactive surfaces', as used herein, includes but is not limited to, for example, —OH, —CO, and —COOH functional groups. Nail polish compositions of the present invention exhibit no agglomeration. Compositions of the present invention generally comprise within the range of about 0.01% to about 20% by weight of oxide nanometric particles which particles are devoid of detectable active/reactive surfaces and which particles can have a mean particle size less than about 10 nm. Particles employed in compositions of the present invention have no reactive groups on the surface and are not subject to surface treatment(s) otherwise known in the art. The nanoscale particles, for example, confer to the compositions of the present invention a viscosity not more than about 1.5 times the viscosity of the composition otherwise without particles. Particularly, the affinity of the nanometric particles employed in compositions of the present invention for other polymer elements of the composition is greater than the affinity of the particles for each other. This resulting effect creates valuable properties of compositions of the present invention including the creation of a dispersion of the particles in the composition without agglomeration. A resulting homogeneous dispersion of particles in the liquid composition leads to an excellent dispersion of the composition upon application and resulting dry film on the nails. Properties of the compositions described herein result in a much improved compromise of hardness in relation to flexibility as well as improved scratch resistance without loss of a valuable gloss effect.

Nanoscale particles for employment in compositions of the present invention are preferably oxides, including but not limited to oxides of zinc, aluminium, titanium, cerium, silicon, manganese, bismuth, copper, zirconium or iron, or a homogeneous mixture of oxides, as mixed oxides of silicon and aluminium. The particles may comprise one or more carbides such as silicon carbide and/or nitrides such as silicon nitride, aluminium nitride or boron nitride. Particles for use in compositions of the present invention are manufactured to have no reactive groups on the surface. This significant property permits the particles to disperse in the general compositions described herein of polymers and organic solvents as generally used in nail polish industry; now however, avoiding problematic agglomeration of the particles. Example particles for use in compositions of the present invention have no hydroxyl groups on their surface. U.S. Pat. No. 5,749,937, for example, incorporated herein by reference, combined with common knowledge in the art of oxides, including but not limited to oxides of zinc, aluminium, titanium, cerium, silicon, manganese, bismuth, copper, zirconium or iron, homogeneous mixtures of oxides, and mixed oxides of silicon and aluminium, describes how to make particles for use in compositions of the present invention. In the case of mixed oxides, products obtained by this way are homogeneous. Particles described herein are commercially available from NanoProducts, Longmont, Colo., USA, for example.

Example nanometric particles for use in compositions of the present invention are generally colourless oxides of aluminium, zinc, zirconium and/or silicon. Preferably homogeneous particles comprise mixed oxides comprising 1 to 99% by weight of aluminium oxide or zirconium oxide or titanium oxide and 1 to 99% by weight of silicon oxide. Particles used in the compositions of the patent are more preferably homogeneous mixed oxides comprising from 10 to 70% by weight of aluminium oxide and from 30 to 90% by weight of silicon oxide. The hardness of these particles, expressed in Moh, is generally greater than about 5 and preferably greater than about 6.

Particles for employment in compositions of the present invention preferably have a refractive index proximate to the refractive index the mixture of polymers used otherwise in the nail polish compositions to yield maximum transparency. Typically, the refractive index for particles of mixed oxides of aluminium and silicon is between about 1.4 and about 1.8, preferably between about 1.5 and about 1.6, depending upon the size of the particles. For a multi-purpose use, colourless particles are chosen; however, coloured particles are used for special effects in particular shades. The particles referred to herein are preferably spherical in shape and have a mean particle size less than about 50 nm, preferably less than about 20 nm calculated with surfaces obtained by electronic microscopy. The range of the size of these nano scale particles may be monomodale or multimodale, but a sharp monomodale range is preferable.

Compositions of the present invention for polishing fingernails comprise about 0.01% to about 5% by weight, preferably about 0.5% to about 5% by weight of the particles described herein. Compositions of the present invention may be coloured or not. The compositions may be applied in one or several coats to nails as an adhering primer or as an upper coat coloured or not.

Preferred compositions of the present invention comprise, for example:
a main film forming polymer, preferably nitrocellulose;
a second film forming polymer, preferably a polyester resin or an acrylic resin;
one or several plasticizers;
one or several solvents;
about 0.5 to about 5% by weight of particles smaller than about 80 nm which particles are devoid of reactive groups and which particles are not surface treated.

“Devoid of reactive groups”, as used herein, is expressly intended to denote substantially devoid of reactive groups (i.e., minimal or negligible amount of reactive groups) for practical purposes of the present invention.

“Surface treated (or treatment)”, as used herein, refers to treatments known in the art including but not limited to dimethylpolysiloxane treatment, dimethicone treatment, polyethylene oxide treatment, and/or treatments otherwise known in the art with one or more synthetic surfactants.

Example plasticizers for use in compositions of the present invention include, but are not limited to, organic citrates such as acetyl tributyl citrate, acetyl triethyl citrates, tributyl citrate and the like; organic adipates such as dioctyl adipate, di isobutyl adipate and the like; organic phosphates such as tributyl phosphate, triphenyl phosphate and the like; glyceryl esters such as triacetin, glyceryl tribenzoate and the like; organic benzoates such as benzyl benzoate, sucrose benzoate and the like; sucrose acetate isobutyrate, camphor, ethyl toluene sulphonamide; and similar structural and functional entities known in the art.

Example solvents for use in compositions of the present invention include, but are not limited to acetates such as ethyl-, propyl-, butyl-, and isobutyl acetates; ketones such as acetone, methyl ethyl cetone, methyl isobutyl cetone and the like; alcohols such as ethanol, propanol, isopropanol, butanol, and the like; ethers as propylene glycol, methyl ether and the like; linear or branched aliphatic hydrocarbons as heptane, cyclohexane and similar structural and functional entities known in the art.

Different compositions of nail polish are prepared, comprising basic components and different quantities or types of nano-scale particles. See, Example 1.

### EXAMPLES

### Example 1

With reference to Table 1, N° 0 is the sample without nano scale particles described herein.

N° 1 and 2 are examples with two different mixed oxides of aluminium and silicon from NanoProducts, Longmont, Colo., USA. There is 2% by weight of these oxides in the composition (1.2% by volume relative to the volume of dry polymer). The first product has a Al/Si atomic % of 6/95 with surface area superior to 100 m²/g (BET method) and a size inferior to 30 nm (Equivalent spherical diameter calculated from surface area). The second product has a Al/Si atomic % of 44/56 with a surface area superior to 30 m²/g (BET method) and a size inferior to 80 nm. The hardness of the two particles is between 7 and 9 Moh.

The example N° 3 is similar to N° 1 with 4% by weight of Al/Si 6/95 particles.

The example N° 4 is similar to N° 1 with a higher level of plasticizer.

The example N° 5 is a comparative trial of a contemporary nail polish product with a colloidal silica (12 nm in size).

TABLE 1

| | Example N^{o} | | | | | |
|---|---|---|---|---|---|---|
| Materials % weight | 0 | 1 | 2 | 3 | 4 | 5 |
| Nitrocellulose (70% in isopropanol | 17 | 16.6 | 16.6 | 16.3 | 15.7 | 16.6 |
| Polyester (70% in butylacetate) | 8 | 7.8 | 7.8 | 7.7 | 8.3 | 7.8 |
| Acetyl tributyl citrate | 6 | 5.9 | 5.9 | 5.8 | 6.2 | 5.9 |
| Ethyl acetate | 28 | 27.4 | 27.4 | 26.9 | 27.4 | 27.4 |
| Butyl acetate | 38 | 37.3 | 37.3 | 36.3 | 37.3 | 38.5 |
| Stearalkonium hectorite | 1 | 1 | 1 | 1 | 1 | 1 |
| Red 34 Ca lake | 2 | 2 | 2 | 2 | 2 | 2 |
| Al/Si 6/95 particles (40% in BA) | — | 2 | — | 4 | 2 | — |
| Al/Si 44/56 particles (40% in BA) | — | — | 2 | — | — | — |
| Colloidal silica | — | — | — | — | — | 0.8 |
| Results | | | | | | |
| Viscosity (a) | 845 | 921 | 954 | 982 | 880 | 1842 |
| Raise of viscosity | x 1 | x 1.11 | x 1.13 | x 1.16 | x 1.04 | x 2.17 |
| Hardness (b) | 202 | 223 | 226 | 234 | 205 | 231 |
| Initial gloss (c) | 82 | 79 | 80 | 81 | 85 | 75 |
| Final gloss (d) | 60 | 75 | 78 | 78 | 70 | 65 |
| Flexibility (e) | 3 | 4 | 4 | 4 | 5 | 4 |

All percentages are % by weight.

Results from the examples indicated in table 1 are describe below:
(a) Viscosity are given in centipoises measured on a Brookfield apparatus with spindle N° 3 and speed 60 turns/nm at 20° C. Below the raise of viscosity is the proportion with the viscosity of example N° with no additives.
(b) The hardness is given in seconds measured on a Persoz pendulum with a 100μ wet film, dried 24 hours at room temperature on glass.
(c) The same film with the same conditions with a 60° gloss meter.
(d) The residual gloss of the same film after a scrubbing with 6000 scans of brush.
(e) Aspect of the film after torsion on a conical mandrel (5: good, 0: significant cracks).
   Results

The nano-scale particles confer no significant raise of viscosity.

There is no aggregation.

With the same quantity of colloidal silica (example 5), the viscosity is more than twice what it was without silica.

The nano-scale particles confer higher hardness by a load effect. However, there is no loss of flexibility. The film is more resistant to elongation. The nano-scale particles provide the effect of an inert extender.

The particles give an initial gloss slightly smaller that ucan be compensated by a higher percentage of plasticizer (example 4).

The films, loaded with the particles described above, have a good resistance to abrasion. Accordingly, the resulting nail polishes exhibit excellent wear and gloss retention.

Additional plasticizer may be added to the composition(s) to yield a more flexible and adherent finished film to nails following application to avoid chipping, wearing and loss of gloss.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described compositions and modes for carrying out the invention which are obvious to those skilled in the art or related fields are intended to be within the scope of the following claims.

1. A nail polish composition comprising about 0.01% to about 20% by weight of non-aggregated particles which have a mean size between about 5 nm and about 100 nm, wherein the particles do not have reactive groups on a surface.

2. A composition according to claim 1 characterized by a viscosity less than about 1.5 times the viscosity of the same composition formulated without the particles as a component.

3. A composition according to claim 1 which comprises about 0.01% to about 5% by weight of the particles.

4. A composition according to claim 3, comprising a main film forming polymer, a secondary film forming polymer, at least one plasticizer, at least one solvent; and, about 0.5% to about 5% by weight of the particles which have a mean size between about 10 nm and about 80 nm, wherein the particles have a refractive index between about 1.4 and about 1.7.

5. A composition according to claim 4 wherein the particles comprise at least one metal oxide wherein the metal is selected from the group consisting essentially of zinc, aluminium, titan, cerium, silicon, manganese, bismuth, copper, zirconium and iron.

6. A composition according to claim 1 wherein the mean particle size is less than about 20 nm.

7. A composition according to claim 1 wherein the particles are homogeneous mixed oxides comprising 1 to 99% by weight of aluminium oxide and 1 to 99% by weight of silicon oxide.

8. A composition according to claim 7 wherein the particles are homogeneous mixed oxides comprising 10 to 70% by weight of aluminium oxide and 30 to 90% by weight of silicon oxide.

9. A composition according to claim 1 wherein the particles have a refractive index between about 1.4 and about 1.7.

10. A method of using a composition for nail polish which composition comprises about 0.01% to about 20% by weight of non-aggregated particles which have a mean size between about 5 nm and about 100 nm, a main film forming polymer, a secondary film forming polymer, at least one plasticizer, at least one solvent, wherein the particles do not have reactive groups on a surface, and wherein the particles have a refractive index between about 1.4 and about 1.7.
